(19) United States
(12) Patent Application Publication
(10) Pub. No.: US 2008/0262351 A1
(43) Pub. Date: Oct. 23, 2008

(54) Microbeamforming Transducer Architecture

(75) Inventor: Steven A. Scampini, Bedford, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/576,401

(22) PCT Filed: Sep. 22, 2005
(86) PCT No.: PCT/IB05/53133

§ 371 Date: Mar. 30, 2007

Related U.S. Application Data
(60) Provisional application No. 60/614,716, filed on Sep. 30, 2004.

(51) Int. Cl. G01S 15/89; G01S 7/521; A61B 8/14; G06K 9/36

(52) U.S. Cl. 600/443; 600/447; 600/459; 367/7; 367/95; 382/131

(57) ABSTRACT

A method for ultrasound imaging utilizes microbeamforming within a transducer probe in electrical communication with a base ultrasound system. The transducer elements are arranged in sub-arrays or subsets, and the transducer includes a cross-point/summation switch in communication with each sub-array, and the base ultrasound system. In the microbeamforming operation, the signals received at the receiving elements comprising a sub-array are summed to generate a composite sub-array signal for same sub-array, and a set of composite sub-array signals corresponding to a particular receive beamforming pattern is defined using a signal controlling the output of the cross-point switch.

This invention relates to medical ultrasound imaging systems and, in particular, to a novel microbeamforming transducer architecture for generating preferred beamforming patterns while minimizing transducer/ultrasound system connections (required channels), and method for implementing a microbeamforming operation based on such an architecture.

Ultrasonic imaging systems use ultrasonic acoustic waves to observe the internal organs of a subject. The frequency range of the ultrasonic acoustic waves typically extends from about 20 kHz (roughly the highest frequency a human can hear) through 15 MHz. The acoustic waves are emitted from the ultrasound systems in a form of ultrasonic pulses, which are echoed (i.e., reflected), refracted, or scattered by structures in the body. These echoes, refractions, and back-scatterings are received by the ultrasound system, which translates them into images, which can be seen on a system display and interpreted by medical personnel.

FIG. 1 depicts a conventional ultrasound system that includes an ultrasonic transducer assembly (referred to in the art as “ultrasound transducer”, “transducer probe” or “scanhead”) 100. The transducer probe is held by the ultrasound system operator and moved over different portions of a subject or patient's anatomy in order to obtain the desired image. Conventionally, ultrasonic transducers assemblies such as transducer probe 100 are connected to the base ultrasound system 130 by a cable 120. The base ultrasound system 130 comprises processing and control equipment 132, as well as the display 133. Those skilled in the art will note that the transducer probe could be readily constructed to include a wireless connection to the base ultrasound system in lieu of cable 120, and the software which drives the beamformer easily modified to receive and process the wireless signals from the transducer probe (e.g., radio transmission; see U.S. Pat. No. 6,142,946, commonly owned and incorporated by reference herein).

The components that transmit and receive the ultrasonic waves in the transducer probe may be implemented differently in various ultrasonic systems. In the ultrasound system of FIG. 1, the face 101 of the transducer probe 100 (which is placed against the flesh of the subject to perform the imaging) includes an array 110 of piezoelectric elements (sometimes referred to as “transducer elements”), which both transmit and receive the ultrasonic waves. In ultrasound systems that use such arrays, the ultrasonic waves are created (and the resulting signals are interpreted) by a process called “beamforming”, which process is performed mostly in signal processing hardware and software. When transmitting, individual piezoelectric elements in the transducer array 110 are stimulated in particular patterns in order to form and focus one or more ultrasonic beams. When receiving, the signal information received by individual piezoelectric elements in the transducer array 110 is delayed, combined, and otherwise manipulated in order to form electronic representations of one or more ultrasonic beams (i.e., beamforming).

One particular known beamforming practice is referred to as multi-line beamforming. In a “multiline” beamforming, the transducer array 110 transmits a single ultrasonic beam, but the receive beamformer electronics synthesize several receive ultrasonic beams with different orientations. The oldest and most basic approach to multiline beamforming is to use multiple single line beamformers that are operated in parallel, such as described in U.S. Pat. No. 4,644,795 to Augustine, which is incorporated by reference. In such an arrangement, each element in the transducer array is connected to a channel of the beamformer. Each of these channels applies delays to the signals from its corresponding element, which delays are appropriate to steer and focus the beam being formed by the beamformer. The signals delayed by each channel of the beamformer are combined to form a uniquely steered and focused beam, and the multiple beams produced simultaneously by parallel operated beamformers are used to form multiple lines of an ultrasound image.

An example of a multiple signal line beamforming architecture is shown in FIG. 2A, in which each of the elements 211 of transducer array 210 (comprising transducer probe 200) has a channel on which any received signals are transmitted over cable 220 to the processing means 232 in base ultrasound system 130. The signals received by the elements 211 may, or may not, be conditioned in the transducer (e.g., impedance matching) and then transmitted over cable 220 to the base ultrasound system. The processing means 232 takes the received signals, which are still in analog form, and converts them to digital signals using analog-to-digital converters (A/D) 233. The resulting digital signals are then delayed by digital delays 234 and summed together by summer 235 to form an acoustic receive sensitivity profile focused at any desired point within an imaging plane.

This approach is sufficient if the number of elements 211 being sampled in the transducer array 210 remains fairly low, i.e., under 200 or so elements (traditional beamformers have 128 channels). If the transducer array 210 has thousands of acoustic elements 211, the particular processing scheme requires that the use of samples from each of those elements, cable 220 would have to carry thousands of channels. Such a scheme would require a prohibitively large cable and more power than is available from a standard electric outlet (the typical power source for most ultrasound systems). For these and other reasons (including the excessive cost of such a cable and the associated electronics), the approach shown in FIG. 2A is not feasible when fully sampling the ˜3000 elements which may be available in a transducer array.

One known solution to this problem of complexity is referred to as “sub-array beamforming” or “micro-beamforming”. One example of a microbeamforming structure capable of implementing a microbeamforming process is highlighted in FIG. 2B. The detailed process is fully described in both the paper entitled “Fully Sampled Matrix Transducer for Real Time 3D Ultrasonic Imaging” by Bernard Savord and Rod Solomon (Paper 3J-1, Proceedings of the 2003 IEEE Ultrasonics Symposium, Oct. 5-8, 2003 (IEEE Press)), and in U.S. Pat. No. 5,318,033 to Savord. Both aforementioned references are incorporated by reference herein. As described in the paper and US patent, and as shown in FIG. 2B, sub-array beamforming requires that the beamforming function be split into two stages, the first stage taking place in the transducer 200, and the second stage taking place in the processing means 232 of the base ultrasound system 130. By performing partial beamforming in the first stage inside transducer 200, the number of channels required to be transmitted over cable 220 to base ultrasound system 130 is drastically reduced.

As shown in FIG. 2B, individual elements 211 in transducer array 210 are grouped into sub-arrays 240-1 to 240-n. Each element 211 in each sub-array 240 has a pre-amplifier 241, and a low power analog delay 242. Each sub-array 240 has a sub-array summer 245 for combining the appropriately delayed analog signals within the sub-array into one channel. Examples of low power analog delay technology which can be used in the first stage include mixers, phase shifters, charge coupled devices (CCD), analog random access memory (ARAM), sample-and-hold amplifiers, and analog filters, etc. All these technologies have sufficient dynamic range and use sufficiently low power to allow their integration into application-specific integrated circuits (ASICs), which ASICs are capable of fitting inside transducer 200 to carry out the microbeamforming application.

When performing microbeamforming, different bulk delays may be applied to each sub-array signal, where each bulk delay imposes the appropriate delay on each sub-array relative to the other sub-arrays. The partially beamformed analog signals from sub-arrays 240-1 to 240-n are transmitted on channels 222-1 to 222-n over cable 220 to processing means 232 in the base ultrasound system 130. The sub-array analog signals are converted to digital by A/Ds 233, appropriately delayed by digital delays 234, and then combined by final summer 235. The bulk delays discussed in the paragraph above may be implemented by digital delays 234.

Although contiguous, the transducer elements, which comprise a sub-array, may form a variety of shapes or patterns on the transducer array. For example, in a rectangularly shaped transducer array, each column of transducer elements may form a sub-array. Such constructions are described in U.S. Pat. No. 6,102,863, U.S. Pat. No. 5,997,479, U.S. Pat. No. 6,013,032, U.S. Pat. No. 6,380,766 and U.S. Pat. No. 6,491,634, each of which are incorporated by reference herein. In the '863 patent, “elevation” beamforming (i.e., combining the signals in each column of elements) is performed in the transducer, while “azimuth” beamforming (i.e., combining the row of previously combined columns) is performed by the processing means in the ultrasound system.

U.S. Pat. No. 6,682,487 teaches that each sub-array forms an irregularly-shaped hexagonal “patch” of twelve transducer elements. As shown in FIG. 3 (a reproduction of FIGS. 6 and 7 of the '487 patent), the transducer array 210 is comprised of small boxes each representing a transducer element 211. The overall transducer array 210 has a roughly dodecahedral circumference, in which the sub-array patches are shown as alternating light and dark groupings. One patch 240 is shown circled in the overall transducer array 210. Patch 240 also is shown magnified above and to the left of transducer array 210. Although shown here spaced apart from each other, the transducer elements 211 (in patch 240) would be closely packed together in a repeating hexagonal pattern. In the transducer array of the '487 patent, the twelve element patch pattern is used only when receiving signals from the subject (i.e., during receive beamforming), whereas a three element pattern is used for transmitting the ultrasonic waves (i.e., during transmit beamforming).

FIG. 4A is a schematic representation of a single analog delay line within a sub-array. As seen in FIG. 4A, the signal received by individual element 211 within sub-array 240 is amplified by pre-amplifier 241 before being appropriately delayed by analog delay 242, which is under the control of control 244. The appropriately delayed signal from analog delay 242 is combined with the appropriately delayed signals from the other elements within sub-array 240 by sub-array summer 245 to form sub-array signals.

FIG. 4B is an exemplary implementation of the single delay line shown in FIG. 4A. As mentioned above, the analog delay may be implemented by any combination of mixers, phase shifters, charge coupled devices (CCD), analog random access memory (ARAM), sample-and-hold amplifiers, and analog filters. The specific implementation shown in FIG. 4B uses analog random access memory (ARAM) to implement analog delay 242. Specifically, the signal received by element 211, after being amplified by pre-amplifier 241, is sampled, i.e., latched onto one of a set of capacitors 420. The sampled signal remains stored on the capacitor until it is latched out of the capacitor (thus applying the appropriate delay). The latched out signal is amplified by post-amplifier 450 before being combined with the other signals in the patch sub-array by sub-array summer 245. The timing of latch-in gates 410 and latch-out gates 430 are under the control of two shift registers 460 and 462, respectively, as part of control 244. Each shift register 460, 462 is arranged to continually circulate one bit, thereby operating as a ring counter. Each bit within shift registers 460, 462 is associated with a corresponding gate in the gates 410, 430. When the circulating one is shifted into a particular bit positioned within the shift register, the gate corresponding to the particular bit latches, resulting in a signal sample either entering or leaving one of the capacitors 420.

Dynamic receive focus module 475 controls the relative timing between when the signals are sampled by latch-in gates 410 and when sampled signals are fed to sub-array summer 245 by latch-out gates 430 (this is used, for instance, to effect a “focal update”). Dynamic receive focus module 475 is under the control of clock delay controller 470, which, in turn, is fed control data for forming the current receive beam from clock command memory 480. Although shown here in a particular configuration, dynamic receive focus module 475 can be placed, and/or implemented, in a number of ways.

FIG. 5 shows a prior art 2D transducer array 210 comprising N rows and M columns of sub-arrays 240 of transducer elements 211. Each sub-array 240 comprises Q rows and P columns of individual transducer elements 211. And in the microbeamforming operation, M×N sub-array receive focusing subsystems 500 are required to transmit each subsystem's summed signal within M×N channels on cable 220. But such a microbeamforming system may nevertheless be improved. That is, it would be a desirable feature in an ultrasound system, which processed utilizing a microbeamforming process to employ additional features, which can achieve arbitrary selection and summation of the microbeamformed or sub-array signals for each of the M×N subsystems comprising the 2D array. For that matter, arbitrary selection of subsystem signals enables implementation of 1D beam patterns at arbitrary angular orientations relative to a central axis of the transducer array, providing valuable data for clinical evaluation.

The inventions disclosed herein rely on the addition of a summation network within the body of the transducer probe to combine outputs of the microbeamforming receive subsystems therein, where the combined outputs are provided to the base ultrasound system as would normally be corresponding beamforming data. That is, by appropriately setting the receive delays within the receive focusing subsystem elements, and by appropriately closing switch elements in the summation network, various receive beam forming patterns can be implemented while requiring significantly fewer receive connections back to the system proper. In a preferred embodiment of the inventive apparatus so described, the summation network may be implemented in a cross-point switch.

It is preferred that the inventive apparatus embody microbeamforming transducer architecture that includes particularly disposed transducer elements which are designated or defined into sub-array groups within the transducer array, and a switch/combiner array within the transducer probe. The switch/combiner array is controlled by an uploaded control signal such that signals from sub-array groups are combined/summed into composite signals and sent to the base ultrasound system for final delay/summing. In another embodiment, a transducer probe built with such 2D array/combiner microbeamforming architecture could readily provide the functionality of a 1D transducer with an electronically rotatable imaging plane and significantly fewer wires back to the system than current 1D architectures (lower cost, better ergonomics, potential for wireless).

Yet another embodiment, the present invention includes a 3D transducer with far fewer wires back to the system than known 3D beamforming systems, where the resulting imaging would have to bear with a somewhat compromised image quality in terms of focusing. Those skilled in the art will readily understand that a continuum of tradeoff exists, which is quantifiable, between number of wires/bandwidth to the system and number of required system front-end channels, and the focusing quality of the system via the beamforming operation (potentially applicable to low-cost systems). This may be particularly attractive to use in catheter based 3D imaging given the tight constraint of return wire bundle diameter.

Moreover, large 3D linears or curved linears, if implemented in accordance with the inventive concepts herein will realize a reduction of cable wire needed, as well as a reduction in system front end costs. The inventions disclosed herein also include a method of implementing the unique abilities of such designed systems, i.e., an ability to implement a microbeamforming process with summation network ability.

The features and exemplary embodiments of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### IN THE DRAWINGS

FIG. 1 shows the large-scale components of a conventional ultrasound imaging system;

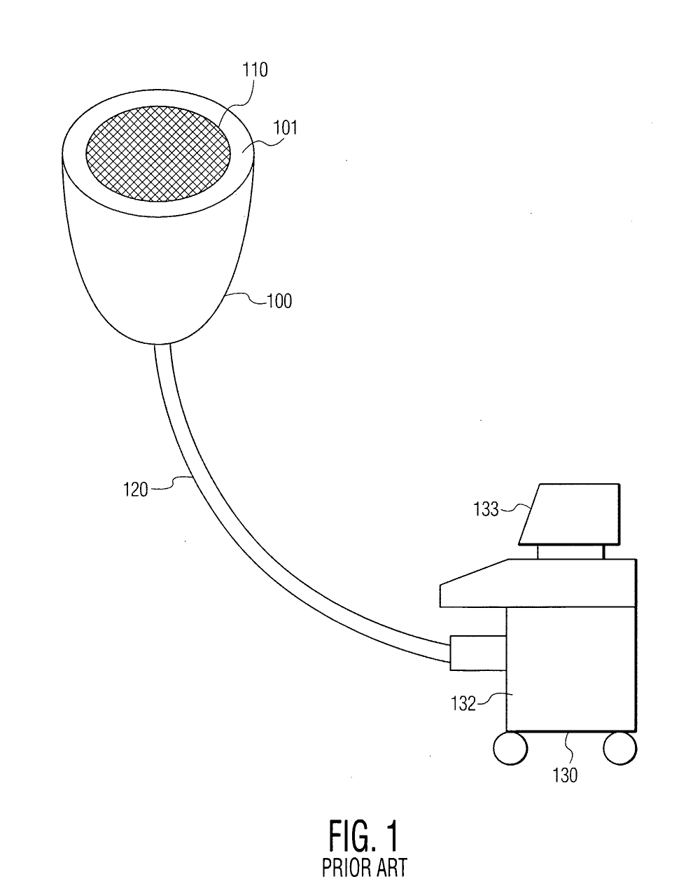

FIG. 2A shows a conventional implementation of multiline beamforming in an ultrasound imaging system;

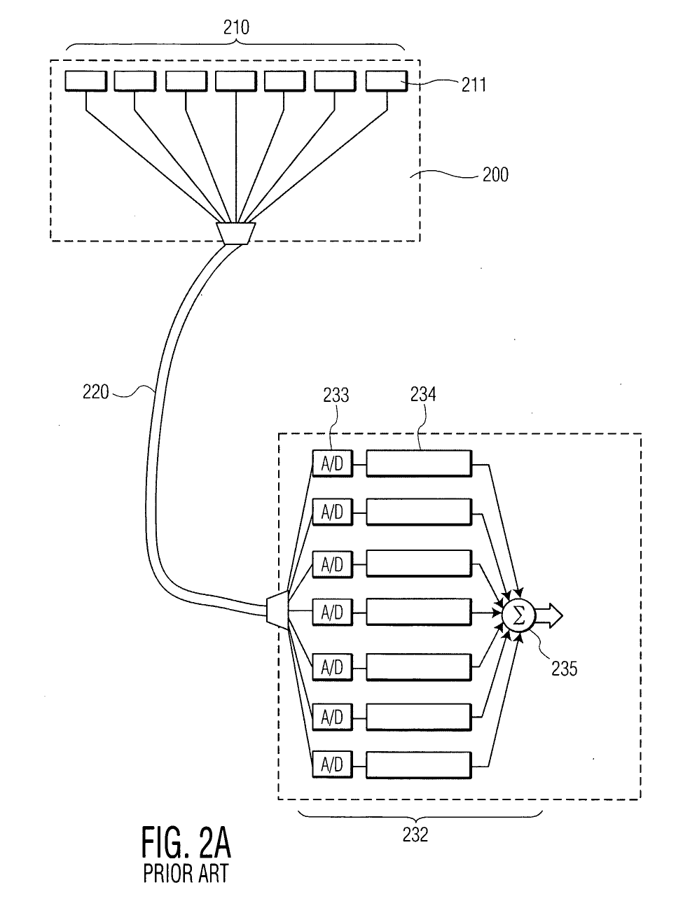

FIG. 2B shows sub-array multiline beamforming according to the prior art;

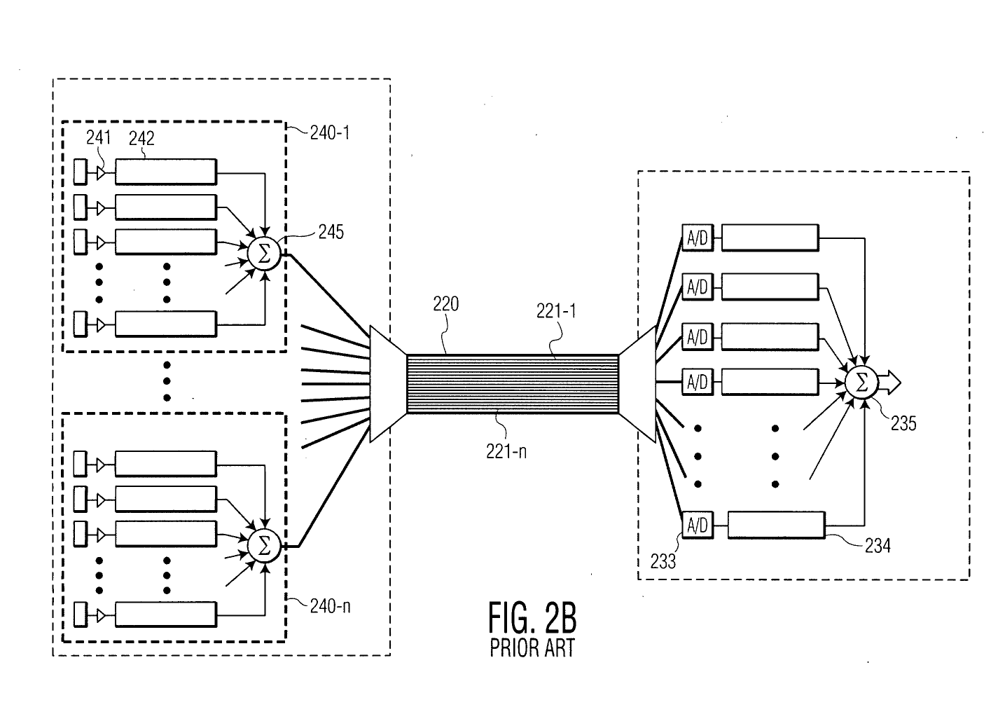

FIG. 3 shows an exemplary embodiment of a transducer array with “patch” sub-arrays for multiline beamforming according to the prior art;

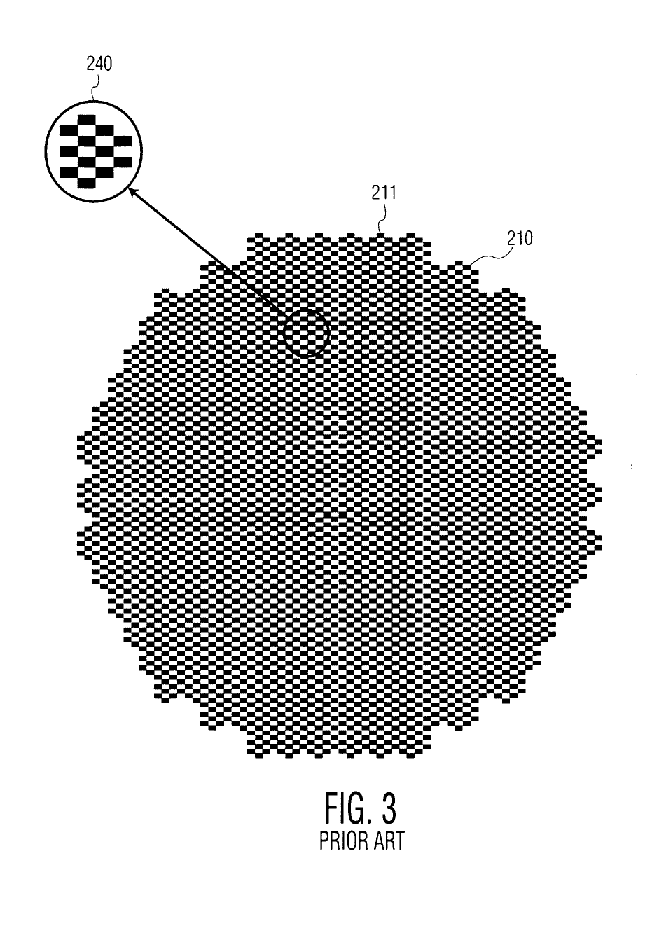

FIG. 4A is a schematic representation of a single analog delay line within a sub-array according to the prior art;

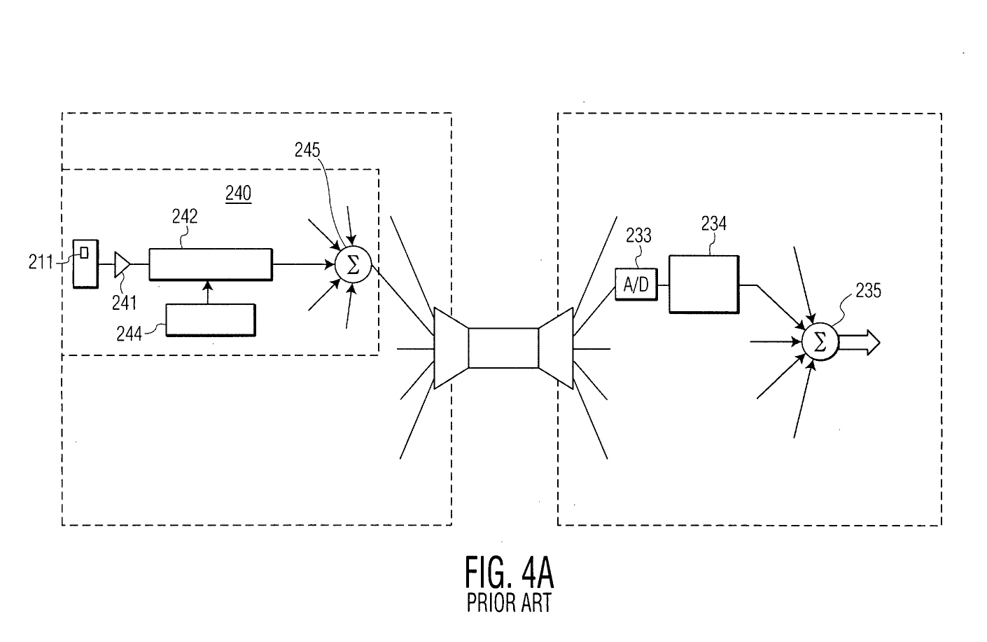

FIG. 4B is a specific implementation of the single analog delay line shown in FIG. 4A using analog random access memory (ARAM) according to the prior art;

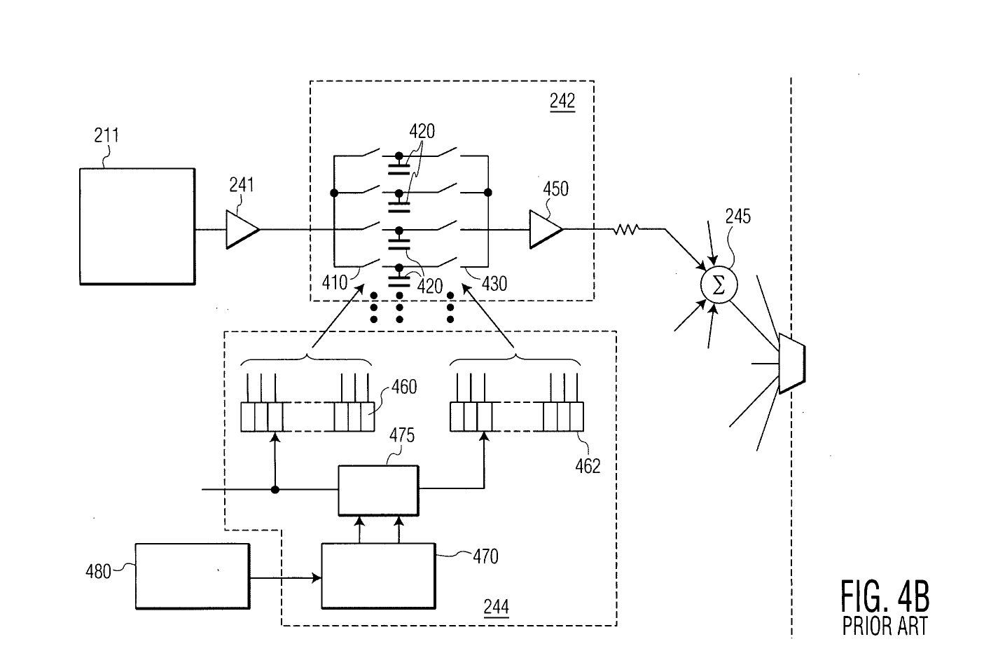

FIG. 5 shows a prior art 2D array constructed to include M×N sub-arrays, where each sub-array comprises P×Q elements, and M×N sub-array receive focusing subsystems;

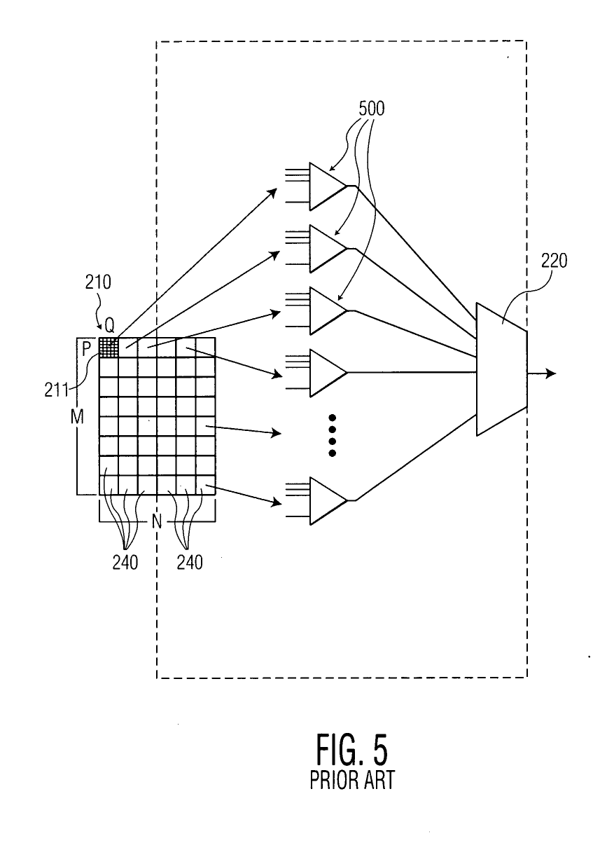

FIG. 6 shows an exemplary embodiment of the transducer sub-array beamforming with crosspoint summation (receive signal path) of the present invention; and

FIG. 7 is a flow chart depicting one process of the present invention.

The inventions described hereafter are applicable to any ultrasound imaging system that uses a transducer probe having a two-dimensional array of individually controllable elements, i.e., piezoelectric elements. The following description is presented in terms of routines and symbolic representations of data bits within a memory, associated processors, and possible networks or networked devices. These descriptions and representations are used by those having ordinary skill in the art to effectively convey the substance of their work to others having ordinary skill in the art. A routine or described processing method embodied in software is here, and generally, intended to be a self-consistent sequence of steps or actions leading to a desired result. Thus, the term “routine” or “method” is generally used to refer to a series of operations stored in a memory and executed by a processor. The processor can be a central processor of an ultrasound imaging system or can be a secondary processor of the ultrasound imaging system. The term “routine” also encompasses such terms as “program,” “objects,” “functions,” “subroutines,” and “procedures.”

In general, the sequence of steps in the routines requires physical manipulation of physical quantities. Usually, though not necessarily, these quantities take the form of electrical or magnetic signals capable of being stored, transferred, combined, compared or otherwise manipulated. Those having ordinary skill in the art refer to these signals as “bits,” “values,” “elements,” “characters,” “images,” “terms,” “numbers,” or the like. It should be understood that these and similar terms are to be associated with the appropriate physical quantities and are merely convenient labels applied to these quantities.

In the present application, the routines, software, and operations are machine operations performed in conjunction with human operators. In general, the invention relates to method steps, software and associated hardware including a computer readable medium configured to store and execute electrical or other physical signals to generate other desired physical signals.

The apparatus of the invention is preferably constructed for the purpose of ultrasonic imaging. However, a general-purpose computer can perform the methods of the invention or other networked device selectively activated or reconfigured by a routine stored in the computer and coupled to ultrasound imaging equipment. The procedures presented herein are not inherently related to any particular ultrasonic imaging system, computer or apparatus. In particular, various machines may be used with routines in accordance with the teachings of the invention, or it may prove more convenient to construct more specialized apparatus to perform the method steps. In certain circumstances, when it is desirable that a piece of hardware possess certain characteristics, these characteristics are described more fully below.

With respect to the software routines described below, those having ordinary skill in the art will recognize that there are a variety of platforms and languages for creating instruction sets for performing the routines described below. Those having ordinary skill in the art will also recognize that the choice of the exact platform and language is often dictated by the specifics of the actual system constructed, such that what may work for one type of system may not be efficient on another system.

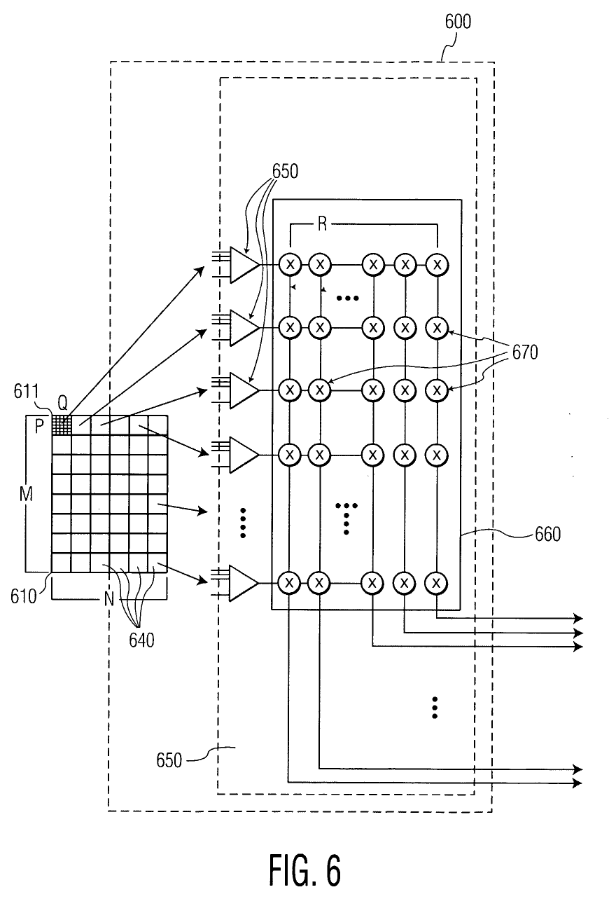

FIG. 6 depicts an ultrasound transducer probe or probe assembly 600 of an ultrasound imaging system of the present invention. The ultrasound transducer probe 600 includes a 2D array 610 of transducer elements arranged in sub-arrays 640, in a form of an M×N matrix or grid of the sub-arrays. The grid, that is, comprises M by N major “patches” of elements, where each patch includes P by Q actual individual transducer elements (611). The P by Q elements 611 for each patch or sub-array are connected to the inputs of a microbeamforming subsystem 650. That is, each of the P and Q elements making up a sub-array is connected to each of M×N sub-array receive focusing subsystems 650 shown in FIG. 6 as part of the microbeamforming subsystem 650. The M times N subsystems cover the entire array. As mentioned earlier, transmit is not shown but in practice, each of these subsystems would include a transmit/receive switch and loadable transmitter. In a practical implementation, the switches for a given “row” might be incorporated within the design of a subsystem cell.

But as distinguished from prior art microbeamforming hardware, where each of the sub-array receive focusing subsystem 650 outputs (each summed sub-array output (signal)) would normally be coupled directly to a base ultrasound system for processing, the sub-array signals generated by the present inventive structure are first processed through a summation network 660, e.g., a crosspoint switch. The summation network 660 enables that outputs of the receive microbeamforming subsystems (sub-arrays 650) may be arbitrarily summed before transfer to a base ultrasound system for use in completing the beamforming operation.

The summation system requires, or preferably includes R×M×N switching elements 670, where R is the number of system receive channel inputs, and M and N are the rows and columns of sub-arrays, respectively. The summing ability of such summation network enables the efficient processing of varied combinations of the sub-array signals from the various sub-array outputs, which varied combinations are efficiently delivered to the base ultrasound system as part of the microbeamforming operation. The resulting beamformed signals made available by these inventions can provide the clinician with data, which would normally be unavailable to the processing hardware and software in the base ultrasound system by use of conventional microbeamforming hardware and software.

In another embodiment, an ultrasound transducer built with this 2D array/summation network architecture in accordance with the invention may be controlled to function as a 1D transducer with an electronically rotatable imaging plane and significantly fewer wires back to the base ultrasound system than current or prior art architectures (lower cost, better ergonomics, potential for wireless). Another embodiment of the invention may be arranged to realizes a 3D transducer with far fewer wires back to the system with somewhat compromised image quality in terms of focusing.

The benefit of the arbitrary summing of the sub-array signals by the combiner/summation network included in the transducer probe is that a continuum of tradeoff between number of wires/bandwidth to the base ultrasound system, and number of required system front-end channels, and focusing quality (potentially applicable to low-cost systems) may be realized (and adjusted to need) by its implementation. So there is great potential applicability to large 3D linear or curved linear arrays, which could greatly improve operation in many ways, e.g., by reducing the number of cable wires and system front-end costs.

While not shown in the figure, those skilled in the art will understand that the transmit control circuitry could be a hardware implementation which is not different or not too different from what is required for operation of Philips current ×4 Matrix transducer probe or assembly. The reader will also note that the control logic and data lines required to load receive delay coefficients and control switch state are not depicted, but may be readily implemented by those skilled in the art in a variety of ways such as serial data lines with shift registers to store values.

An example of use of this architecture is the implementation of a one dimensional beam pattern (1D) at an arbitrary angular orientation relative to the central axis of the transducer by loading the appropriate delays and closing the appropriate switches. The result is arbitrary beam plane selection with significantly fewer return connections than is currently the case in the commercially available Philips ×4 Matrix Live3D transducer.

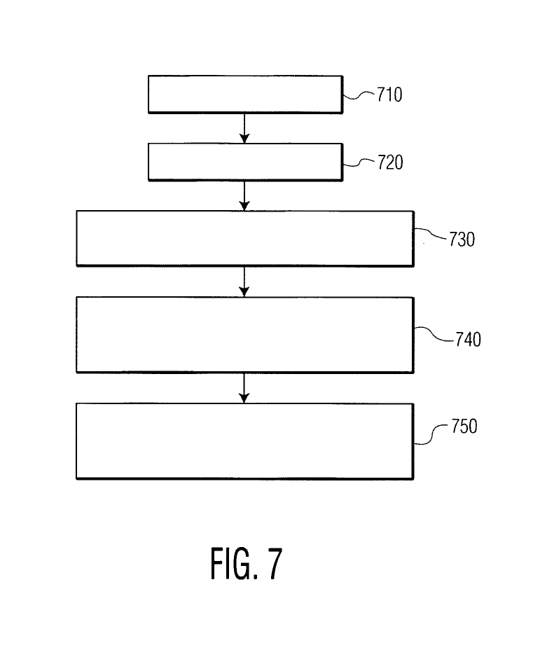

FIG. 7 is a flowchart which depicts an exemplary embodiment of a processing method of the present invention. The inventive ultrasound image processing method utilizes microbeamforming to generate a plurality of microbeamformed sub-array signals, where the sub-array signals may be arbitrarily combined within a transducer probe in electrical communication with a base ultrasound system. Within the base ultrasound system, the arbitrarily combined sub-array signals are further processed to complete a beamforming operation.

Box 710 of FIG. 7 defines a step of transmitting ultrasound signals from an array of transducer elements disposed in the transducer probe into a region of interest in the subject. Box 720 defines a step of receiving signals echoed from the region of interest at the transducer elements, wherein the transducer elements are grouped in sub-arrays of P by Q elements. Box 730 defines a step of focusing all signals (on all P & Q elements) within a sub-array within a sub-array receive focusing subsystem, which may result in the formation of M×N sub-array signals. As mentioned above, each of the sub-array focusing subsystems sum the signals received at the P by Q receiving elements of each sub-array to generate a composite sub-array signal within the transducer probe.

Box 740 defines a step of selecting a predetermined set or composite of sub-array signals, in accordance with a preset or delivered control signal provided to a summation network, where the composite set of sub-array signals corresponds to a particular receive beamforming pattern. And box 750 defines a step of communicating the set or composite of sub-array signals from the transducer probe to a signal processing system for completing the beamforming process.

1. An ultrasound diagnostic imaging system, comprising:
an ultrasound transducer, comprising:
an array of transducer elements for transmitting ultrasound transmit pulses, and receiving echo signals in response to the transmit pulses, the transducer elements arranged in sub-arrays, such that echo signals received by the elements in each sub-array are added/combined to generate a weighted, composite sub-array receive signal; and
a summation/combiner network including an input channel coupled to each sub-array for receiving each composite sub-array receive signal, and for combining/summing a particular selection or subset of the composite sub-array receive signals to realize a desired beamformer pattern; and
a base ultrasound system, comprising:
a processor for processing the particular selection or subset of sub-array receive signals to generate a display signal, which display signal is suitable for causing an output device to produce an image, and
a system controller for controlling the display processor.

2. The ultrasound diagnostic imaging system as set forth in claim 1, further comprising signal transfer means for transferring the particular selection or subset of sub-array receive signals to the base ultrasound system.

3. The ultrasound diagnostic imaging system as set forth in claim 2, wherein the signal transfer means includes a cable including channels corresponding to each sub-array.

4. The ultrasound diagnostic imaging system as set forth in claim 1, wherein the summation/combiner network is a crosspoint switching/summation network.

5. The ultrasound diagnostic imaging system as set forth in claim 1, wherein the ultrasound transducer further includes a receive focusing subsystem for each sub-array.

6. The ultrasound diagnostic imaging system as set forth in claim 5, wherein the receive focusing subsystem includes controllable delay elements for setting receive delays for the elements of each sub-array.

7. The ultrasound diagnostic imaging system as set forth in claim 1, wherein the system processor generates said at least one control signal for controlling the summation/combiner network.

8. A sub-array receive microbeamformer for an ultrasound imaging system which comprises an ultrasound transducer with an array of transducer elements arranged in a plurality of sub-arrays, wherein each sub-array is constructed to generate a composite sub-array signal from the elements comprising the sub-array, a summation network constructed to arbitrarily sum at least two composite sub-array signals to realize various receive beamforming patterns, and means for communicating arbitrarily summed composite signals to a base ultrasound system for further processing.

9. A method for ultrasound imaging which utilizes microbeamforming to generate a plurality of microbeamformed sub-array signals, where the sub-array signals may be arbitrarily combined within a transducer probe in electrical communication with a base ultrasound system, within which base ultrasound system the arbitrarily combined sub-array signals are further processed to complete a beamforming operation, the method comprising the steps of:
transmitting ultrasound signals from an array of transducer elements disposed in the transducer probe into a region of interest;
receiving signals echoed from the region of interest at the transducer elements, wherein the transducer elements are grouped in sub-arrays of P by Q elements;
summing the signals received at the P by Q receiving elements of each sub-array to generate a composite sub-array signal within the transducer probe;
selecting a predetermined set or composite of sub-array signals, where said set or composite of sub-array signals corresponds to a particular receive beamforming pattern; and
communicating the set or composite of sub-array signals from the transducer probe to a signal processing system for completing the beamforming process.

10. The ultrasound imaging method as set forth in claim 9, wherein the step of selecting includes summing particularly selected composite sub-array signals to generate a set of composite sub-array signals for processing by the signal processing system.

11. The ultrasound imaging method as set forth in claim 9, wherein the step of selecting further includes setting receive delays for all of the elements in a sub-array, and for defining a switching pattern for composite sub-array signals using a summation network whereby various receive beamforming patterns may be realized.

12. The ultrasound imaging method as set forth in claim 9, wherein the transducer array utilized is a 2D array and is controlled to operate as a 1D array with electronically rotatable image plane.

13. The ultrasound imaging method as set forth in claim 9, wherein the transducer array utilized is a 3D array.

14. The ultrasound imaging method as set forth in claim 9, wherein the particular combination is determined as a compromise between the number of channels communicating between the transducer and the base ultrasound system, and the desired bandwidth.

15. The ultrasound imaging method as set forth in claim 9, wherein step of communicating includes utilizing a cable.

16. The ultrasound imaging method as set forth in claim 9, wherein step of communicating is implemented wirelessly.

17. A computer readable medium which includes a set of instructions for carrying out the ultrasound imaging method as set forth in claim 9.
